(19) United States
(12) Plant Patent Application Publication
(10) Pub. No.: US 2007/0209094 P1
(43) Pub. Date: Sep. 6, 2007

(54) Syzygium australe named '4tune8one'

(76) Inventor: Graham Fortune, Cooroy (AU)

Correspondence Address: CHRISTIE, PARKER & HALE, LLP, PO BOX 7068, PASADENA, CA, 91109-7068, US

(21) Appl. No.: 11/367,121

(22) Filed: Mar. 3, 2006

(51) Int. Cl. A01H 5/00

(52) U.S. Cl. PLT/263

(57) ABSTRACT

A new a distinct variety of Syzygium australe, which is a periclinal chimera arising from a spontaneous mutation (sport) in a stock plant of a standard green Syzygium australe is described. This new variety comprises a unique variegated leaf color, vigorous growth and compact foliage habit making it suitable for hedging plants, topiary species and tub specimens in domestic, commercial and recreational settings.

### BOTANICAL CLASSIFICATION

Syzygium australe.

### CULTIVAR DENOMINATION

‘4Tune8one’

### BACKGROUND OF THE INVENTION

The present invention relates to a new and distinct variety of Syzygium australe, and herein after referred to by the varietal denomiation of the new species ‘4tune8one’.

Syzygium is a genus of about 500 species occurring in tropical and subtropical rainforests. There are about 50 species in Australia occurring in the Kimberly region of Western Australia, the Northern Territory, Queensland and northern New South Wales. The species formerly known as Eugenia australis is now regarded as two separate species, Syzygium australe and S. paniculatum.

S. australe is a common plant of coastal and highland rainforests from southern New South Wales to central Queensland. In nature it can reach over 25 meters in height but is usually smaller in cultivation. The leaves are opposite (each pair emerges from the stem at the same location but on opposing sides), rounded and bright, glossy green in color. White flowers are followed by large, fleshy, red fruits in profusion. These are ovoid in shape and around 20 mm long with a large seed. The fruits are edible and are often made into jams. They can also be eaten fresh and have a crisp, refreshing texture.

S. australe is a hardy tree in most temperate and subtropical climates and is cultivated in most Australian states. It prefers rich soils and assured moisture. At least two compact forms have been marketed in recent years. These probably grow to no more than 2 meters and may be found under the names “Blaze” and “Tiny Trev”. The compact forms are suitable for growing in large containers.

There is an ongoing need worldwide, and in particular in the United States of America, for bushy, attractive, vigorous growing hedging plants, topiary species and tub specimens. Such plants find use in domestic, commercial and recreational settings such as parks and gardens.

### SUMMARY OF THE INVENTION

4Tune8one is a periclinal chimera resulting from a spontaneous mutation (sport) in a stock plant of a standard green Syzygium australe growing on the property of the inventor in Cooroy, Queensland, Australia. The new variety has been propagated vegetatively through six generations and have shown that the unique features of this new plant are stable and reproduced true to type.

### COMPARISON WITH PARENTS AND COMPARABLE VARIETIES

The most similar variety known to the inventor is the variety ‘Pearly Showers’, the subject of U.S. Plant Pat. No. 5,286, issued Sep. 25, 1984. ‘4tune8one’ differs from ‘Pearly Showers’ in that leaves emerge all year round, have a different leaf color, abundant flower development and persistence, and edible seeded fruit.

The primary features of this new variety which are distinctive over existing types comprise its variegated leaf color, vigorous growth and compact foliage habit. New growth comprises a combination of red hues occurring where the green parts of the mature leaf will eventually be. As the leaves mature the coloration changes to a yellow and yellow-green along the edges, and variable tones of brown-green, yellow-green and green in the centre of the leaf. Leaves show varying degrees of speckled and dappled variegation. Vigorous growth of this new variety includes new leaves emerging all year round, and leaves may persist even when stems mature into branches. The compact foliage habit with a dense foliage crown is observed.

### BRIEF DESCRIPTION OF THE ILLUSTRATIONS

The accompanying photographic illustrations show typical species of the new variety, in color as nearly true as it is reasonably possible to make in a color illustration of this character. Colors in the photograph may differ from the color values cited in the detailed botanical description below, which accurately describes the colors of the new plant species.

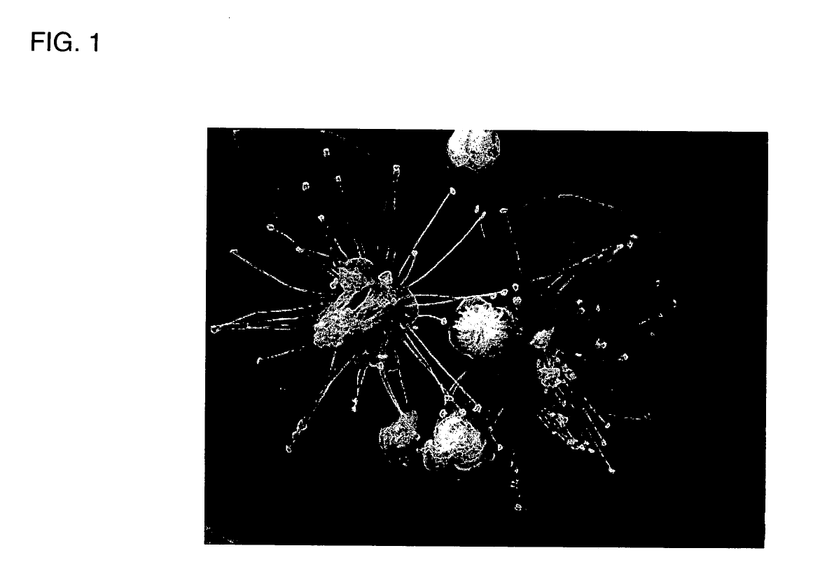

FIG. 1 comprises a close up view of flowers of ‘4tune8one’. Note the white petals and pink-red colored sepals and the unique variegation of each leaf.

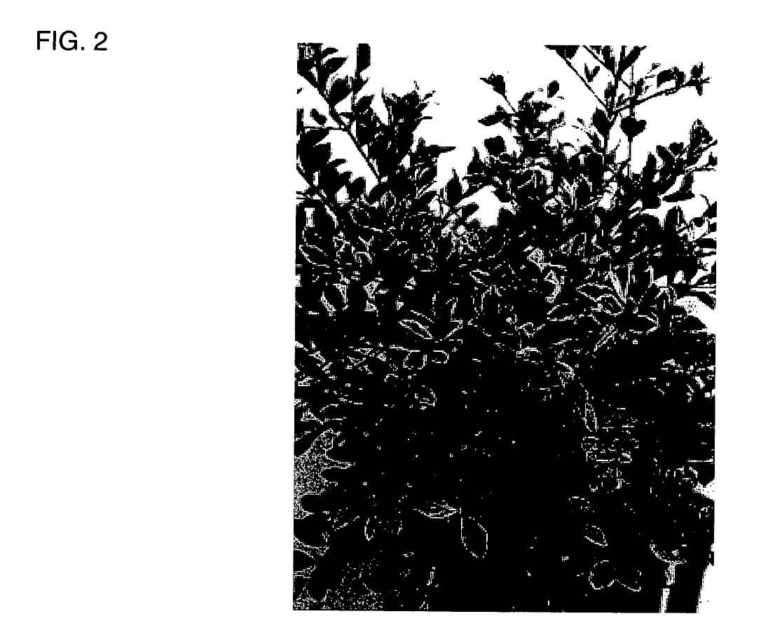

FIG. 2 comprises an overall view of variegated foliage on a 5-month-old plant.

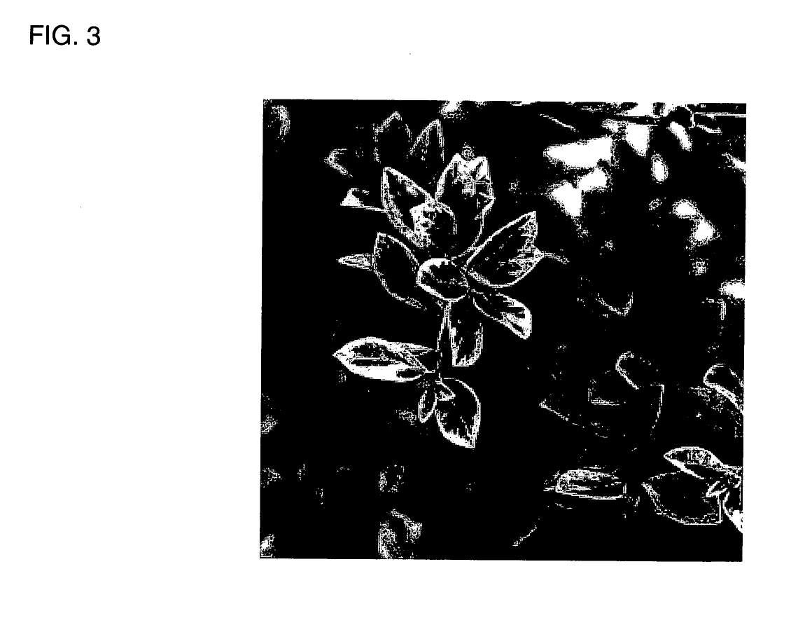

FIG. 3 comprises a close-up view of mature variegated foliage.

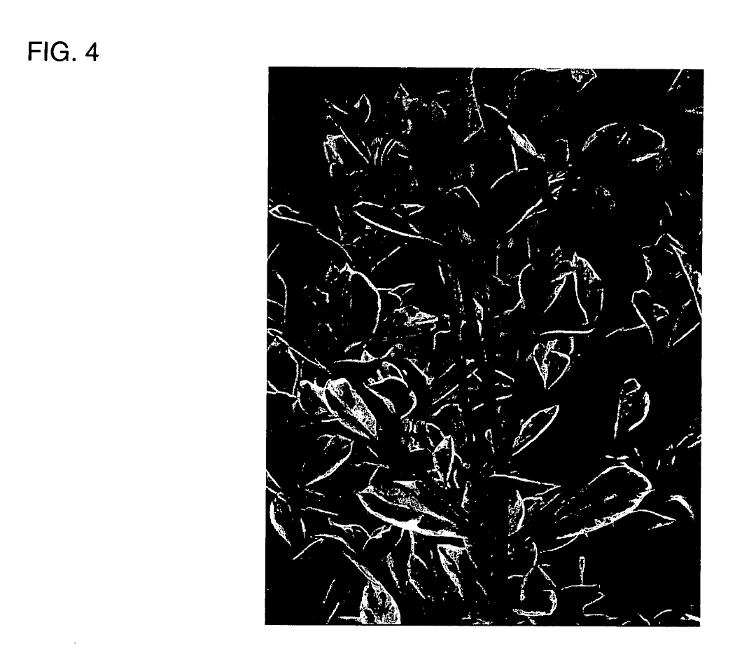

FIG. 4 comprises a view of the scarlet red new growth.

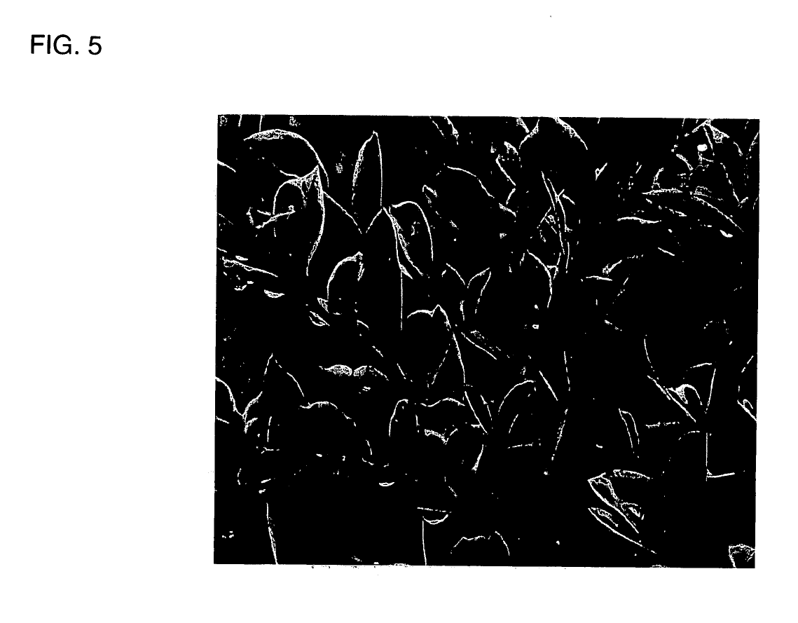

FIG. 5 comprises a view of the color change of the plant as stem matures.

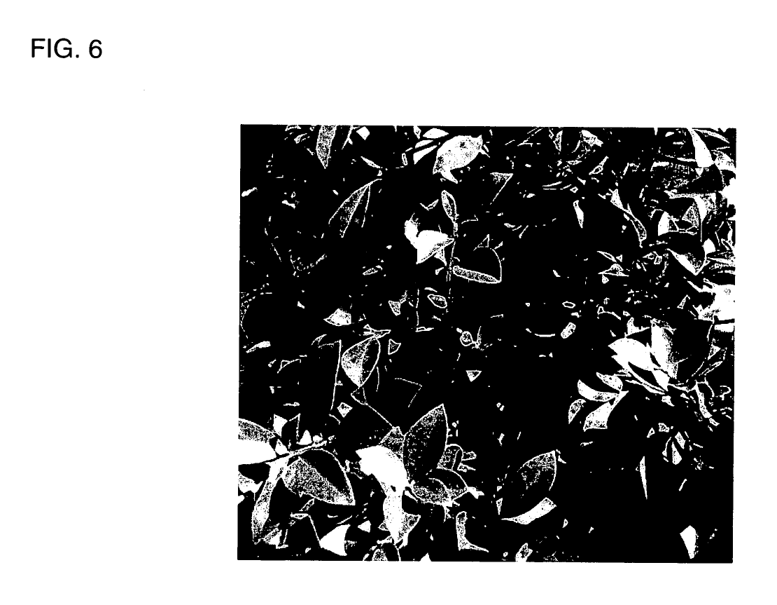

FIG. 6 comprises a view of a mother stock plant with normal green leaves.

### DESCRIPTION OF THE NEW VARIETY

Syzygium australe, ‘4Tune8one’ has not been observed under all possible environmental, cultural, and light conditions. The following observations and descriptions are of plants grown in containers at Cooroy, Queensland, Australia. Phenotypic expression may vary with light intensity, cultural, and environmental conditions. In this description, color is by reference to The Royal Horticultural Society Chart (R.H.S.) in respect of plants, for plants from 5 months to 2 years old.
Propagation:

The new variety developed from a natural sport of a Syzygium australe topiary at the nursery of the inventor, Graham Fortune in Cooroy, Queensland, Australia. The natural sport appeared in January 2003. Seven original sport cuttings were taken from the mother plant by the inventor and propagated on his property. The new variety is readily propagated, with 13,000 plants produced up to February 2006 on the inventor's property.

The new variety has been propagated vegetatively with 13,000 plants produced so far by vegetative propagation (asexual reproduction from stem cuttings). Only three off-types were observed amongst this group demonstrating extreme stability and uniformity.

The new variety is vegetatively propagated from root cuttings, stem sections or by tissue culture.
Plant description:

The new variety is a medium sized, vigorous, single or multi-trunked tree with a dense foliage crown. Ultimate height is unknown, but is presently 10 feet tall and 3 feet wide. It is probable that in cultivation this cultivar will reach a height of 23 feet and width of 5 feet. The non-variegated parent can reach an ultimate height of 70 feet.
Stem and branch description:

Stems and branches of ‘4tune8one’ are disposed opposite each other, upright and 4-angled (0.16 inch×0.12 inch) when young. Internodes of the young stems range from 0.39 inch to 0.71 inch, increasing with age to a maximum internode length of 1.26 inches. The initial color of stems ranges from glossy orange-red RHS 44A through to glossy scarlet-red RHS 45B. The color darkens with age to dull orange-red RHS 53A as shown in FIGS. 4 and 5. Young stems have a longitudinal internodal scar/ridge in line with the base of the petiole where leaves are attached to the stem, they vary in color from red RHS 46A to bright red RHS 45C. The internodal scar/ridge are not visible after stems reach a diameter of 2.36 inches. As stems age and thicken the color changes to a dull yellow-green RHS 150C undertone with a greyed-yellow RHS 160C overtone. Branches will form from the main trunk/s from ground-level up.
Leaf description:

Leaves of ‘4tune8one’ are simple, shiny and shaped ovate to elliptic with young tips of leaves being acute. Mature leaves can be up to 1.7 inches long and 0.91 inch wide, with petioles being up to 0.24 inch long. The leaves have entire margins and are arranged in opposite decussate pairs. Leaves are normally carried on 45° angles to the stem/branch. Leaves can be persistent even when stems mature into branches. New leaves emerge all year round, which is unusual for the species and compared to the cultivar Syzygium paniculatum ‘Pearly Showers’ U.S. Plant Pat. No. 5,286.
New foliage:

The new growth comprises one or a combination of the following red tones: RHS 53A, RHS 53B, RHS 47A, RHS 46B, RHS 45A, RHS 45B. These colors occur where the green part of the leaf will eventually be. The red pigmentations are especially vivid during cooler months.
Mature foliage:

As leaves mature the color is yellow-green RHS 153C, RHS 153D, RHS 151A, RHS 151B, RHS 151C, RHS 151D, RHS 18C and RHS 11C along the edges and variable tones of color ranging from brown-green RHS 152B, yellow-green RHS 144A and green RHS 137A in the centre. All leaves show varying amounts of speckled and dappled variegation in each leaf. Some leaves can have up to 5 combinations of the above mentioned colors or can appear to only have two. Each leaf has a unique form of variegation as depicted in FIGS. 1-5. As leaves mature the darker green areas of the lamina appear to have a slightly raised appearance in contrast to the light areas of the variegation which appear sunken and slightly distorted.
Inflorescence/reproductive organ description:

Flowers and fruit of ‘4tune8one’ appear in early summer and continue through to early autumn. The flowers are 0.59 inch to 0.83 inch wide with numerous conspicuous whitish (RHS 155D) stamens (as shown in FIG. 1) that are 0.51 inch to 0.83 inch long. The flower has four whitish (RHS 155D) petals which are 0.16 inch to 0.24 inch long free and spreading; four obtuse sepals which are initially green (RHS 141D) and change color to RHS 48C with age to a final mature color of RHS 53C (as shown in FIG. 1). The flowers appear in small clusters/panicles of 3 to 5 flowers with pedicels up to 0.39 inch long, the hypanthium is campanulate in shape and 0.2 inch long. The flowers appear throughout the foliage of the plant towards the ends of branches giving a showy appearance. The fruits that form are fleshy and single seeded and are obovoid in shape, 0.59 inch to 0.98 inch long by 0.31 inch to 0.79 inch wide that range in color from dark pink RHS 55A, deep pink RHS 54A and red RHS 53A. The fruits have persistent sepals. The edible fruits ripen through autumn to winter.

Winter growth of ‘4tune8one’ is comparable with other Syzygium australe and Syzygium paniculatum.
Disease/drought resistance description:

Under conditions of limited water availability the new variety revives quickly on the application of water.

In general, ‘4tune8one’ has been observed to be no more susceptible to pests and diseases than other commercial varieties.

1. A new and distinct variety of Syzygium australe plant, substantially as shown and described.
